(19) United States
(12) Patent Application Publication
(10) Pub. No.: US 2005/0137657 A1
(43) Pub. Date: Jun. 23, 2005

(54) Apparatus and method for emitting light to a desired target location

(76) Inventor: Douglas R. Dykaar, Waterloo (CA)

Correspondence Address: TEITELBAUM & MACLEAN, 1187 BANK STREET, SUITE 201, OTTAWA, ON, K1S 3X7, CA

(21) Appl. No.: 11/016,901

(22) Filed: Dec. 21, 2004

Related U.S. Application Data
(60) Provisional application No. 60/531,628, filed on Dec. 23, 2003.

(51) Int. Cl. A61N 5/06

(52) U.S. Cl. 607/88

(57) ABSTRACT

An optical device for irradiating a target with light is disclosed wherein an optical waveguide has a first span or portion with a first end for being coupled with a light source. The first span has a diameter suitable to guide and support light having an electromagnetic mode coupled therein without substantial optical loss. The first span has a region which tapers to a second span or portion of the optical waveguide. The second span is formed in a coil which guides light poorly wherein optical loss is substantial. The second span having a diameter less than the first span. In operation, light radiates outward from the second span along its length. Preferably, the coil has an inner diameter of less than 1 cm.

### CROSS-REFERENCE TO RELATED APPLICATIONS

This application claims priority of U.S. Provisional Patent Application No: 06/531,628 filed Dec. 23, 2003, entitled “Apparatus for Photo-dynamic Therapy and Method for Manufacturing Same” which is incorporated herein by reference for all purposes.

### FIELD OF THE INVENTION

This invention relates to a device and method for use as a light emitter and or probe where light is to be routed and delivered to a desired target location and more particularly a preferred embodiment of the invention relates to a light source suitable, for example, for use with compounds for Photo-Dynamic Therapy (PDT) of a target tissue or compositions in a mammalian subject.

### BACKGROUND OF THE INVENTION

In PDT it is desired to controllably illuminate tissue to activate a targeted substance. Photo-dynamic therapy (PDT) is a relatively new approach to treating many cancers. Patients are injected with one or more photosensitive drugs, such as one known as Photofrin, that bind to the rapidly dividing cells. A narrow-band laser is then used to excite the drugs, inducing a reaction which kills the cells. PDT has been used to treat esophageal cancer, Kaposi's sarcoma, an AIDS related condition, and the overgrowth of blood vessels in the eye (macular degeneration), which afflicts seven million people in North America alone.

Some PDT techniques treat skin lesions and subcutaneous tumors by exciting them with UV lamps. Notwithstanding, many PDT techniques require the use of lasers to provide the high power light at a target distal from the laser. The light emitted from the laser must be captured and highly guided until it reaches the target location where it must efficiently and uniformly exit the fiber and irradiate the target. For many applications, optical fibers are required to direct the light to the desired location, with a fiber tip constructed to uniformly couple the light from the fiber into the surrounding tissue.

The prior art in this field includes tips of various designs such as diffusive tips shown and described in U.S. Pat. No. 5,151,096, 5,169,395; diffusive loops as are shown in U.S. Pat. No. 5,632,737, and reflective surfaces cut or etched into the optical waveguide are described in U.S. Pat. No. 5,496,308. The abovementioned patents are incorporated herein by reference. Although these approaches attempt to achieve their intended function, they have limitations, both in terms of ease of fabrication, mechanical robustness, and scalability to small dimensions.

It is a first object of this invention to provide an improved method and apparatus for delivering electromagnetic radiation to a desired region.

It is a further object of this invention to provide a low cost and less complex optical radiator for use in PDT and other therapeutic applications.

As many existing techniques for emitting light along a span or length of a fiber do not simply scale to small sizes, it is the object of this invention to provide a light emitter capable of suitably illuminating and irradiating a target with light and method for constructing such a device.

In a primary application of PDT in accordance with this invention, Watts of optical power are emitted along a length of an optical fiber anywhere from less than one cm to more than 10 cm.

While the described invention applies to single-mode fibers, it is applicable to other types and sizes of fibers and waveguides as well. Notwithstanding, below diameters of approximately 1 micron, the propagation of the light in the fiber changes to a purely surface mode and is not suitable for this application.

### SUMMARY OF THE INVENTION

In accordance with an aspect of this invention an optical device for irradiating a target with light is provided, comprising: an optical waveguide having a first end for being coupled with a light source, the first end having a diameter suitable to guide and support light having an electromagnetic mode coupled therein substantially without optical loss to a second span of optical waveguide with a diameter smaller than the electromagnetic mode of light propagating therein, so that in operation, light radiates from the second span of the optical waveguide, wherein the second span of optical waveguide is formed in coil of at least one turn having an inner diameter of less than 10 mm.

In accordance with another aspect of the invention an optical waveguide is provided for directing light to a target location, comprising a first section having a mode field diameter sufficient to support and guiding light launched therein received from a light source, and having a second section contiguous therewith formed from the same waveguide wherein the mode field diameter is smaller than the mode field of light launched into the first section, the second section being tightly wound around a mandrel; having diameter of less than 1 cm.

### BRIEF DESCRIPTION OF THE DRAWINGS

Exemplary embodiments will now be described in accordance with the drawings in which:

FIG. 1 is a drawing in accordance with this invention illustrating an optical fiber being heated and pulled.

FIG. 2 is shows the pulled fiber of FIG. 1 having two different diameters across cross sections along lines A-A and B-B.

FIGS. 3 and 4 illustrate a fiber being heated and wound on a mandrel.

FIG. 5 is a detailed view of a fiber wound on a mandrel.

FIG. 6 illustrates an embodiment wherein a fiber wound on a mandrel is included within a laser cavity.

FIG. 7 shows an embodiment of the invention wherein a standard single mode fiber is wound tightly around a mandrel having a diameter of less than 1 cm in accordance with an embodiment of the invention.

FIG. 8 illustrates an embodiment of the invention wherein a coiled optical fiber is protected by having epoxy contacting the inner fiber coil.

FIG. 9 illustrates an alternative embodiment of this invention wherein a coiled optical fiber emitter is within a protective light transmissive housing.

FIG. 10 is an embodiment of the invention having a tapered mandrel.

FIG. 11 illustrates an embodiment of the invention wherein a taper is provided in the optical fiber resulting in the reduction in the optical fiber diameter, which increases the radiative loss due to mode coupling into the cladding.

FIG. 12 is a diagram wherein the mandrel and resulting structure is hooked.

### DETAILED DESCRIPTION

This invention relies on a process of inducing guided light in the core of an optical fiber to radiate out of the fiber. This has been accomplished in the past in systems other than optical fibers, for example in microwave waveguides. An important aspect of this invention, is to reduce the dimension of the optical waveguide, for example, the optical fiber, to a point at which the electromagnetic mode is no longer well guided in the waveguide. In a microwave guide the energy that is not guided is coupled into the metal conductor of the guide and absorbed. In an optical waveguide the energy is coupled into the cladding. This has been used in the past to construct energy absorbing “dumps” for signals in both the electrical and optical domains where no reflections are desired. This is described on the Internet at http://www.yet2.com/app/insight/techofweek/26458?sid=230 “New Termination Process Enables Cost-Effective Manufacturing of Tunable Optical Fibers”, Northrop Grumman,”

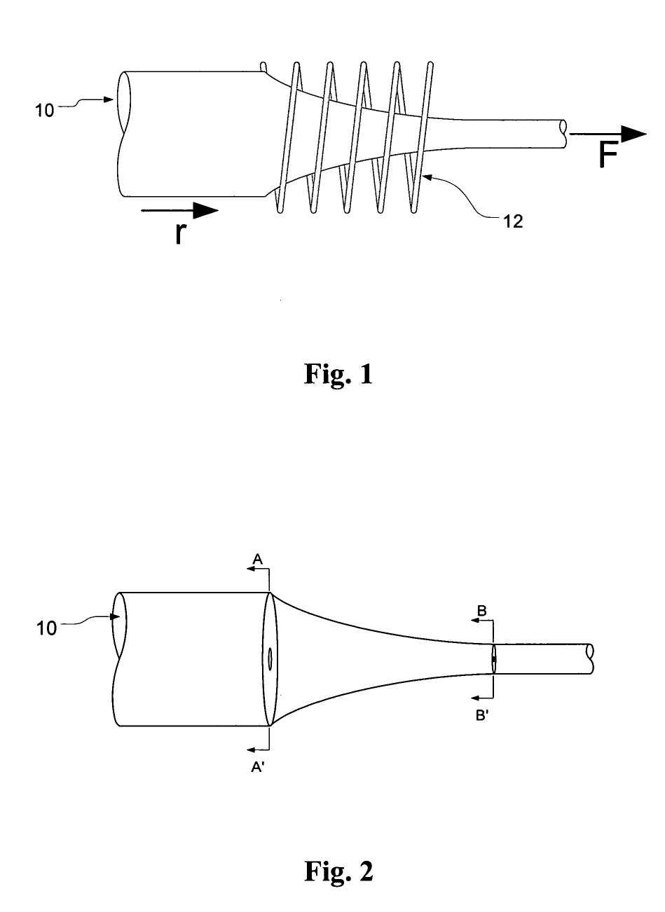

Referring now to FIG. 1 an optical fiber 10 is pulled by heating a section thereof by any convenient means, such as a heater, 12, as shown, or alternatively by a flame, laser, or other means. The fiber 10 is pulled with a force, F, and fiber is fed into the heater 12 at a rate, r. This allows the fiber to be pulled in much the same way as the fiber was originally pulled from the pre-form as fibers are typically made. It is of note that a fiber pre-form is generally inches in diameter, wherein the fibers used in accordance with this invention are mm scale or smaller. The change in the propagation characteristics of the fiber comes from the reduction in size of the waveguide, relative to the wavelength. In a preferred embodiment of the invention the core of the optical waveguide or fiber 10 is reduced to a size where the optical mode no longer fits in the core. When a significant fraction of the optical mode extends outside the effective core, light will advantageously couple out along the length of the fiber. By carefully controlling the diameter of the pulled fiber, the leakage characteristics can be controlled. For small diameters, the leakage/unit-length can be high enough to be useful for applications such as PDT. Referring now to FIG. 2, the cross section at A-A′ of the fiber 10 is shown, schematically indicating the core size in relation to the cladding. In cross section B-B′, the pulled fiber 10 is shown to have a reduced core diameter. The ratio of diameters, D is constant:

$$\frac{{D}_{core}}{{D}_{cladding}} = {[ \frac{{D}_{core}}{{D}_{cladding}} ]}_{pulled}$$

For single-mode fiber, the core diameter is so small relative to the mode size, the core and cladding combined becomes the effective core of a step-index fiber, where the new cladding has the index of the material surrounding the fiber. For multi-mode fiber, the pulled diameter may be larger, but the leakage will still occur for diameters that are small relative to the mode size in the unperturbed fiber.

There are two aspects that must be considered for this invention to be practical. The first is the direction of the emitted light. Light coupled from the core is still propagating nearly parallel to the direction of the fiber. For PDT type applications one desires a more uniform angular (Lambertian) distribution along the length of the fiber.

The second aspect is mechanical rigidity. Optical fibers are not rigid to begin with and after reducing the diameter to the point where significant light leakage is occurring, the mechanical rigidity is insufficient for PDT type applications or other application where the fiber must be inserted into tissue, or otherwise operate unsupported.

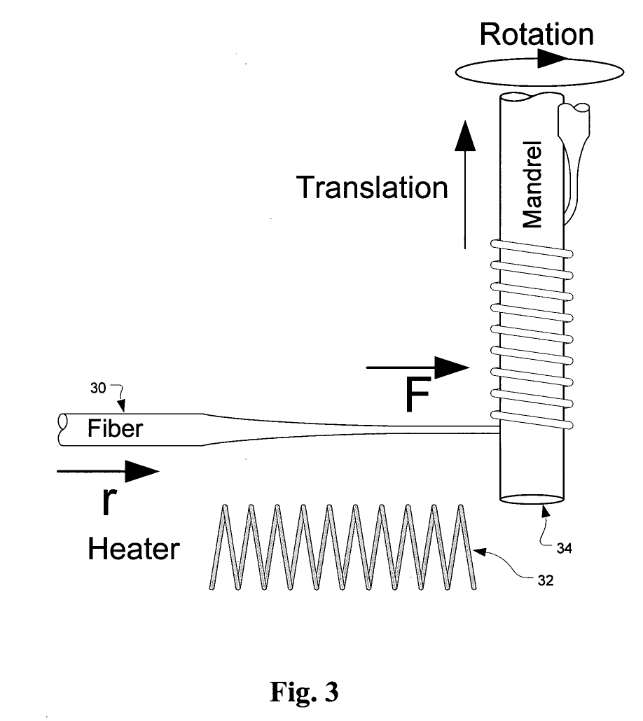

Both of these shortcomings can be overcome simultaneously by winding the pulled fiber onto a mandrel 34, using the winding configuration shown schematically in FIG. 3. A heater 32 is shown offset. Separate heaters can be used for melting the fiber 30, and keeping the fiber 30 softened as it is wound on the mandrel 34. The mandrel 34 can be used to provide the force, F in FIG. 3 through the application of a torque on the mandrel 34. This allows the pulled fiber 30 to be supported immediately upon being formed, while still softened from heating. It also allows the fiber 30 to adhere to the mandrel 34 after cooling.

Note that the curved fiber will have enhanced radiative losses. The radiative loss, αᵣ is:

$${α}_{r} ∝ exp [ \frac{- R}{{R}_{0}} ]$$

Large losses can be defined to occur beyond a critical radius of curvature.

For Single mode fibers, R_{Csingle} is:

$${R}_{Csingle} \approx \frac{20 {λ ( 2.748 - 0.996 \frac{λ}{{λ}_{c}} )}^{- 3}}{NA}$$

For Multi-mode fibers, R_{Cmulti} is:

$${R}_{Cmulti} \approx \frac{3 {n}_{1}^{2} λ}{4 π {NA}^{3} \sqrt{{n}_{1}^{2} - {n}_{2}^{2}}}$$

As is evident from these equations, the fiber need not be as thin for a given level of radiative loss if it is also curved beyond the critical radius. For typical fibers, the critical radius is less than 1 cm.

The mandrel advantageously also provides the necessary mechanical rigidity, and wire diameters <100 μm to serve as the mandrel 34 are commercially available. Mandrel materials may be non-ferrous, such as ceramic or sapphire, as suits the particular application. The winding process also changes the orientation angle, of the fiber 30 relative to the original propagation direction anywhere up to 90° to the original axis of propagation.

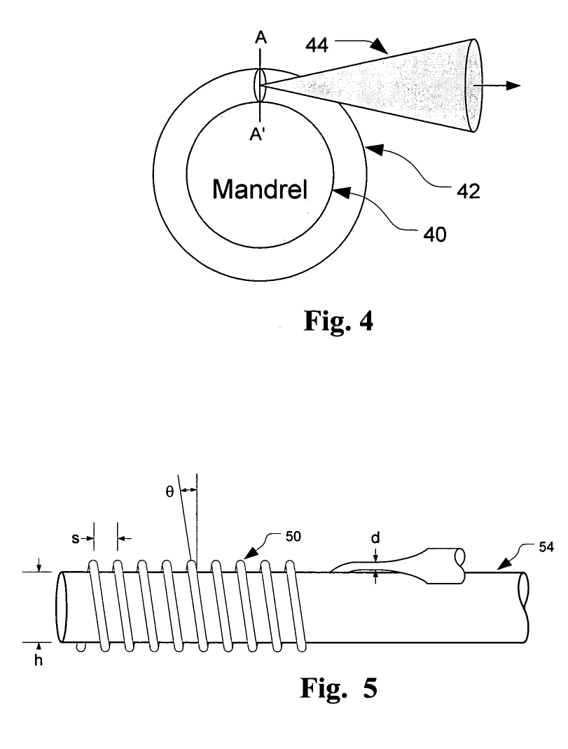

FIG. 4 shows a cross section of the mandrel, 40, and fiber, 42. The cross section A-A′ of the fiber, 40, radiates into the surrounding medium, approximating a cone, 44.

The end of the mandrel can be shaped or pointed to allow insertion of the device into the target tissue. The mandrel 34 can be provided with a loop, hole or other shape or tube to capture the input end of the fiber. The mandrel 34 can also be curved so that the wound fiber assembly also exhibits curvature.

As shown in FIG. 5, by controlling the diameter d of the fiber 50, the angle θ, of the fiber 50 relative to the mandrel 54, the spacing s between windings, the diameter of the mandrel 54, h, one has enough parameters to design a desired emission pattern. Note that while not shown in FIG. 5 any of the variables can vary along the length of the device. As an example, the fiber diameter may be kept large during the first turn, where the fiber 50 changes orientation relative to the mandrel 54.

All of the parameters listed above can be controlled independently though the use of the mandrel 54 as a take-up spool for the pulled fiber 50, by controlling the heater temperature(s), mandrel position, mandrel torque and fiber feed rate. Fracturing the fiber at the mandrel (e.g. by cooling and pulling or bending) terminates the winding process.

In addition the fiber can be doped, or otherwise loaded with scatterers, or coated to enhance or otherwise tailor the emission pattern. U.S. Pat. No. 5,908,415 incorporated herein by reference entitled Phototherapy Methods and Apparatus, issued Jun. 1, 1999 in the name of Sinofsky discloses a light delivery system wherein light scatterers are used to scatter the light within an optical fiber. The mandrel can be shaped into convenient shapes, textured to aid the winding process, coated to enhance reflectivity or absorption, or otherwise processed without deviating from the present invention.

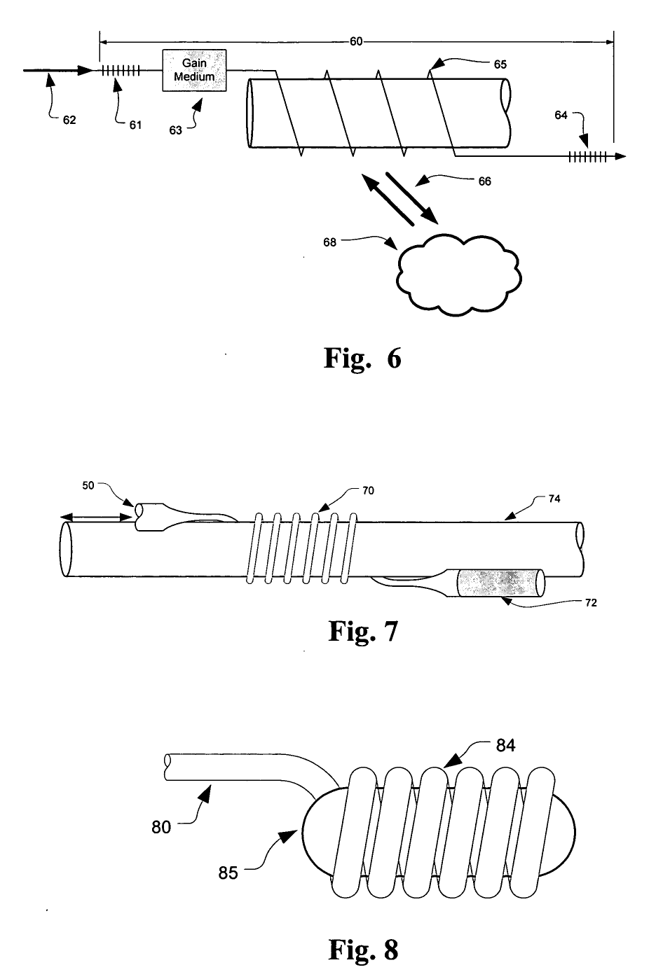

The same device can be used to couple light into the fiber, from the surrounding medium, as a detector of light (e.g. tissue fluorescence). As an example, the fiber can contain wavelength conversion dopants (e.g. Er). The fiber-coil can also be made a part of a laser cavity 60, as shown in FIG. 6. The cavity consisting of dichroic mirror, 61, allowing pump laser 62, to pump gain medium 63, and output coupler 64.

The fiber coil interacts, 66 with the surrounding environment, 68. The interaction, 66, is an exchange of photons which need not be the same wavelength. Detectors external to the laser (not shown) can monitor the back-reflected light and can be used to monitor the device performance. This technique is also applicable to the other geometries.

Advantageously, incorporating a coiled-fiber non-linear element 65 inside a cavity takes advantage of the how the cavity-Q is affected by the element. Proper design and construction the intra-cavity element can induce a very large effective gain in the output of the laser; for example if the element is lossy enough to suppress lasing, and then couples light into the cavity at a wavelength in the gain-bandwidth of the laser. The cavity embodiment shown in the figure is exemplary and other configurations can be implemented, such as, for example a loop configuration.

In the embodiments described heretofore, in accordance with the invention, a light emitter has been shown wherein a length of waveguide or optical fiber was heated and pulled, so as to have a smaller diameter in the light-emitting region.

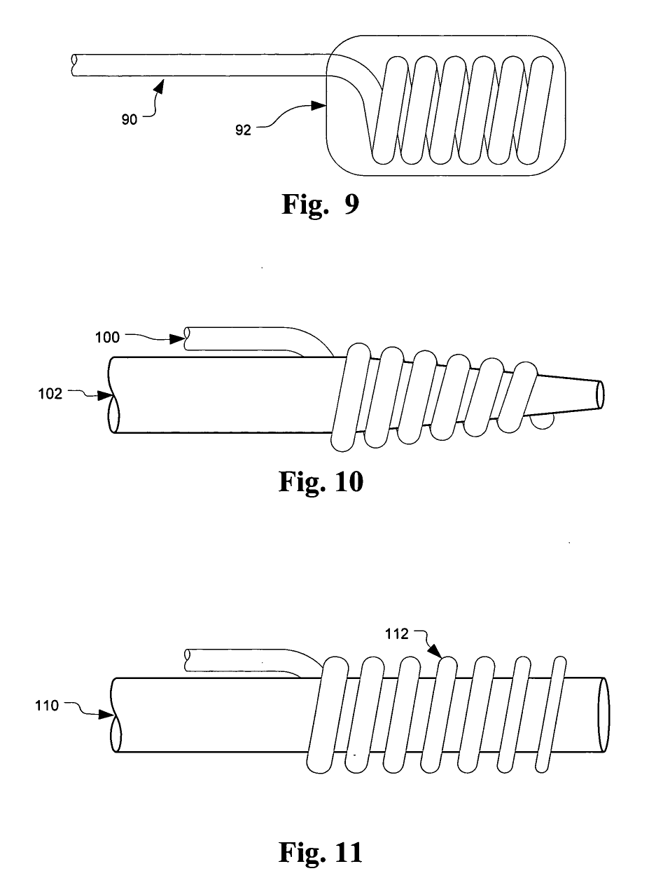

FIGS. 7, 8 and 9 illustrate alternative embodiments wherein a single or multimode optical fiber is coiled in a coil having a small diameter for example, less than 1 cm, and preferably smaller to serve as a light emitter. The use of epoxy in FIG. 8, or a light transmissive protective housing in FIG. 9 are also applicable to embodiments shown in FIGS. 1 through 6.

Turning now to FIG. 7, a length of optical fiber 50 having a section with 6 turns around a mandrel is shown. The diameter of the mandrel 74 is less than 1 cm thereby providing a “lossy” wound region where light launched into the fiber leaves the core and propagates outward into the cladding and then to the surrounding environment as it becomes unguided by the bent fiber. The term “coiled fiber” is to connote a fiber having at least one 360-degree turn, and not merely a fiber having a slight bend. As in other embodiments described above, the mandrel 74 serves as a tool on which the optical-fiber coil 70 may be wound, and additionally serves as a stiffener, protecting the otherwise delicate optical fiber from damage. Also shown in FIG. 7, an end of the fiber opposite form an end where light is launched into the fiber 50 has a reflector, 72 at its end face to reflect backwards any light that has not been emitted from the coiled emitter section. Light reflected backwards can then be emitted when traversing the coil in a reverse direction. The reflector can be, but is not restricted to, a fiber grating or cleaved facet.

FIG. 8 illustrates an embodiment of the invention wherein epoxy 85 is used to contact adjacent coils 84 of the fiber 80 to serve as a stiffener. An alternative protective means is shown in FIG. 9, where the coiled fiber is placed in a light transmissive capsule serving to protect the coiled emitter from damage. In any of the embodiments described in accordance with the invention a termination reflector can be provided to recirculate light backwards that was not emitted outward from the coiled section.

In FIG. 9, an embodiment is shown where the mandrel is removed and the fiber coil 90, is encapsulated or otherwise potted in an optical material 92. The encapsulating material 92 may have advantageous optical properties such as enhanced scattering or contain dopants or other optically active materials.

Turning now to FIG. 10, an alternative embodiment of the invention is shown wherein tapered mandrel 100 is provided which allows the curvature to increase, the radius of curvature of the fiber 102 thereby decreasing with length along the mandrel 94. As the curvature of the optical fiber 102 increases, the leakage increases. However the optical power is decreasing with length as light leaks out. The taper therefore allows the power coupled out of the fiber to be kept constant along the length of the mandrel.

Combinations of the available parameters shown in FIG. 5 and FIGS. 7-11 of fiber diameter, mandrel diameter and taper, fiber-coil spacing and the presence or absence of a reflector on the end of the fiber coil can be used to design an element having the desired optical distribution as a function length along the axis of the device.

In FIG. 11, for a mandrel, 110 of constant diameter, the reduction in fiber diameter, 112 increases the radiative loss due to mode coupling into the cladding. Both of these embodiments can be combined using a tapered mandrel and a tapered fiber to achieve a desired radiation pattern along the length of the mandrel. Of course the embodiments of FIG. 10 and 11 may also be combined having a tapered fiber on a tapered mandrel.

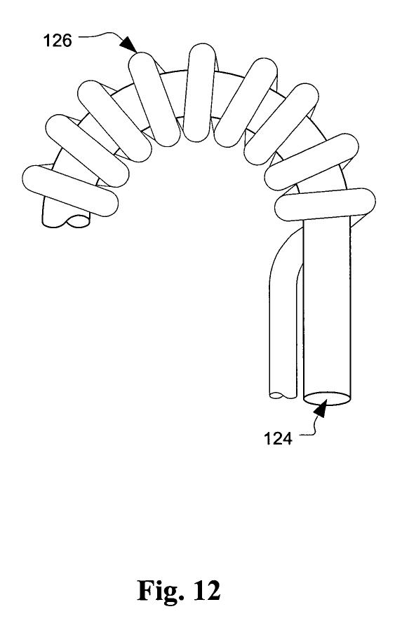

Referring now to FIG. 12 an embodiment is shown using a bent or shaped mandrel 124 to allow radiating light preferentially in a desired direction from the fiber, 126. The direction of radiation can be further controlled by coating part of the mandrel (e.g. the outer radius) with a reflective or scattering material to direct the light towards the center of the mandrel bend. The bend can be shallow relative to bending radius required to induce radiation loss in the fiber as the radiation loss due to bending is induced in the perpendicular plane. The hook shape of the bent mandrel may be useful for radiating into a small tumor without having to puncture it.

Although the embodiments described heretofore have been directed to a device for irradiating a target with light other embodiments may be envisaged where an optical fiber on a mandrel as described could be used as a sensor, where light is only interacting with the surface, or sensor material on the surface. In this instance an optical fiber on a mandrel designed to be less susceptible to bending losses could be used as a sensor. Lessening bending losses could be achieved by using specialized fiber optimized to reduce bending loss, e.g. so-called “holey” fiber, or fibers coated with reflective coatings. Optical fiber of this type is described in a paper entitled “Development of Holey Fiber Supporting Extra Small Diameter Bending” by Nishioka et al., in Information and Communication Systems, SEI Technical Review number 58, June 2004, pages 42-47, incorporated herein by reference.

Of course numerous other embodiments may be envisaged without departing from the sprit and scope of the invention.

1. An optical device for irradiating a target with light comprising:
a first optical waveguide with a first end for being coupled with a light source, the first span having a diameter suitable to guide and support light having an electromagnetic mode coupled therein without substantial optical loss;
a second optical waveguide optically coupled to the first optical waveguide, wherein in operation the second optical waveguide guides light propagating therein poorly such that optical loss is substantial, wherein the second optical waveguide is formed in a coil forming at least a single loop of 360 degrees, the coil having an inner diameter of about 1 cm or less so that in operation, light radiates outward from the second span along its coiled length.

2. An optical device as defined in claim 1, wherein the first optical waveguide is an optical fiber and wherein the second optical waveguide is an optical fiber.

3. An optical device as defined in claim 2 wherein the first optical waveguide and the second optical waveguide are contiguous, being formed of a same optical fiber.

4. An optical device as defined in claim 3, wherein the second optical waveguide is coiled about a mandrel.

5. An optical device as defined in claim 3, wherein the coiled second optical waveguide has a plurality of turns and is provided with at least one of a mandrel or light transmissive jacket for protecting the coiled second optical waveguide from damage.

6. An optical device as defined in claim 3 wherein the coiled second optical waveguide has a medium contacting adjacent coils to stiffen the coils.

7. An optical device as defined in claim 1 wherein the second optical waveguide has a smaller diameter than the first optical waveguide.

8. An optical waveguide as defined in claim 1 further comprising a reflector at or near an end of the second optical waveguide for reflecting light backwards.

9. An optical waveguide as defined in claim 3 further comprising a reflector at or near an end of the second optical waveguide for reflecting light backwards.

10. An optical device for irradiating a target with light comprising:
an optical waveguide having a first span with a first end for being coupled with a light source, the first span having a diameter suitable to guide and support light having an electromagnetic mode coupled therein without substantial optical loss, the first span having a region which tapers to a second span of the optical waveguide formed in a coil which guides light poorly wherein optical loss is substantial, the second span having a diameter less than the first span, wherein in operation, light radiates outward from the second span along its length, and wherein in the coil has an inner diameter of less than 1 cm

11. An optical device as defined in claim 10, wherein the optical waveguide is an optical fiber and wherein second span is contiguous with the first span, and wherein the second span is formed in a coil by heating and pulling the waveguide around a mandrel.

12. An optical device as defined in claim 10 wherein the second span of optical waveguide is formed in coil about a mandrel, having a diameter of less than 1 mm.

13. An optical waveguide device as defined in claim 10, wherein the optical waveguide is an optical fiber and wherein the first span and the second span are contiguous and wherein the second span has a plurality of coils disposed within a protective light transmissive housing.

14. An optical device for irradiating a target with light as defined in claim 1 wherein the second optical waveguide and the first optical waveguide are a same optical fiber and wherein the second optical waveguide is formed in a coil having a plurality of turns with an deceasing radius along a length thereof.

15. An optical device as defined in claim 10 wherein the coil has a decreasing radius.

16. An optical device as defined in claim 4, wherein the mandrel along a length thereof.

17. An optical device as defined in claim 1, further comprising scatterers within the waveguide for scattering light.

18. An optical device as defined in claim 1, wherein a gain medium in the presence of a pump source is in optical communication with the first or second optical waveguides so as to provide energy to the second optical waveguide.

19. An optical device comprising an optical fiber fixedly wound on a mandrel into at least one single coil of 360 degrees wherein the bend radius of the optical fiber is less than 1 cm.

20. An optical device as defined in claim 10, wherein the second span has a diameter less than a diameter of an electromagnetic mode of the light propagating therein.
